(19) United States
(12) Patent Application Publication
(10) Pub. No.: US 2020/0132668 A1
(43) Pub. Date: Apr. 30, 2020

(54) FLOW-THROUGH QUANTITATIVE BLOOD COLLECTION VIAL

(71) Applicant: DAXOR CORP., New York, NY (US)

(72) Inventors: Jonathan Feldschuh, Jackson Heights, NY (US); Ronald Cacheiro, Oak Ridge, TN (US)

(73) Assignee: DAXOR CORP., New York, NY (US)

(21) Appl. No.: 16/667,965

(22) Filed: Oct. 30, 2019

Related U.S. Application Data
(60) Provisional application No. 62/753,295, filed on Oct. 31, 2018.

(51) Int. Cl. G01N 33/49; A61B 5/15; G01N 1/14

(52) U.S. Cl. CPC G01N 33/4915; G01N 1/14; A61B 5/150992; A61B 5/150099

(57) ABSTRACT

A device is presented for storing, transporting, measuring, and collecting blood, having the properties of a precisely determined volume, the ability to be emptied or filled while connected to a continuous source (such as a supply of a drug, or the bloodstream of a patient), and a geometry suitable for the entire device to be placed in the counting chamber of a detector (such as the counting well of a gamma counter).

### CROSS-REFERENCE TO RELATED APPLICATIONS

This application claims the benefit of U.S. Provisional Patent Application No. 62/753,235, filed on Oct. 31, 2018, the contents of which are herein incorporated by reference in their entirety into the present application.

### FIELD OF THE INVENTION (TECHNICAL FIELD)

The present invention relates to systems and methods for storing, transporting, measuring, and collecting blood.

### SUMMARY OF THE INVENTION

A device is presented for storing, transporting, measuring, and collecting blood, having the properties of a precisely determined volume, the ability to be emptied or filled while connected to a continuous source (such as a supply of a drug, or the bloodstream of a patient), and a geometry suitable for the entire device to be placed in the counting chamber of a detector (such as the counting well of a gamma counter).

### BACKGROUND

Many forms of quantitative vials for fluid collection exist. These are generally designed to collect very small quantities of fluid, which are then measured by some means. The present invention deals with the situation where the vial is designed to be fillable in a flow-through manner; where the contents can be measured by a counting apparatus at any time, including continuously as fluid moves through the vial; and where the volume of fluid to be collected is at least 0.5 ml (i.e. greater than the approximate maximum of 0.15 ml using capillary action). The use of a precise geometry for the vial facilitates applications where measurements of a sample (collected or monitored in the vial) are compared with measurements of a reference standard (contained in an identical vial). An example of such an application would be the measurement of an unknown volume using the indicator dilution method, whereby a known amount of tracer is introduced into the unknown volume; the amount of tracer present in a sample collected from the unknown volume can be directly compared to the amount of tracer present in a reference standard created by diluting the same tracer into a known volume, and filling an identical vial with the resulting dilution.

Blood is a complex fluid, comprising a watery plasma containing various proteins (principally albumin) and large cells (principally red blood cells). Quantitative collection of blood must deal with a variety of factors specific to blood: the potential for clotting (especially when exposed to air); the potential for hemolysis (when the fluid is disturbed or agitated excessively); the potential for settling (when blood is allowed to stand under the influence of gravity); the potential for surface adhesion (the proteins in blood are generally “sticky” and will adhere to most plastic surfaces, and will resist displacement under typical flow conditions); the potential for non-laminar flow caused by partial separation on the blood components caused by the geometry of the blood pathway through the vial. As a result of these issues, quantitative measurements of blood that require volumes greater than 0.15 ml generally involve a cumbersome, multi-step process. First blood is collected (e.g. into a vacutainer tube at the bedside) and then it must be transferred quantitatively into a separate container (e.g. using a precision pipette at a laboratory bench). Pipetting of whole blood is quite difficult and potentially inaccurate, because of all the issues mentioned above (of settling, adhesion, hemolysis, etc.). Centrifuging whole blood to get access to plasma is feasible, but this adds time and complexity to a quantitative process, and still requires skill in pipetting to ensure accuracy (e.g. to pipette only plasma and no red cells). For all these reasons a solution enabling direct collection of precise amounts of whole blood directly from a patient for the purposes of quantitative measurement is desirable.

### BRIEF SUMMARY OF EMBODIMENTS OF THE PRESENT INVENTION

In one preferred embodiment, a quantitative flow-through vial is fashioned in a precise, repeatable manner via molding, out a plastic material with suitable handling properties, such that there is a continuous fluid pathway embedded in a retaining structure, with standardized input and output ports, said retaining structure being shaped to fit into a detector (for example, having a test-tube shape matching the dimensions of a counting well in a radiation scintillation detector), with the volume of said vial being precisely known.

In one preferred embodiment, the cross section of the pathway of the blood is transformed from circular (at the ingress and egress, to mate with standard connectors having a circular cross section) to elliptical (to allow for efficient space-filling use of the volume of the vial, particularly in the lower portion of the vial that has the highest counting efficiency) and back to circular.

In one preferred embodiment, the vial is made of two moldable components, an outer container (e.g. in the shape of a sample container for a given well) and an inner component with a fitted cover with ingress and egress, arranged so that there is a narrow fluid pathway down the sides leading to a larger well at the bottom of the assembled vial. This has the advantage of providing a larger volume of fluid, concentrated in the area of maximum counting efficiency.

In one preferred embodiment, the vial is made of one extruded and two moldable components that are fused together: an inner coil, an outer container, and a fitted cover with ingress and egress. The coil is constructed from a material chosen for its desirable flow properties, particularly low surface adhesion of blood proteins.

In one preferred embodiment, the vial is a flat spiral, designed to be placed on a flat detector (or in between two flat detectors). Such a flat vial would have excellent counting stability with respect to settling, as the entire vial would be within the field of view of the detector, and the vertical displacement of blood components within the vial would be very small even with complete separation due to settling.

In one preferred embodiment, the vial can be sealed securely with one-way valves so as to transport a quantitative dose of a drug or tracer, prior to its being precisely administered to a patient via connection in a flow-through manner.

In one preferred embodiment, the vial is pre-treated with a compound to alter the surface adhesion properties of the material such that the flow-through performance of the vial is enhanced when exposed to sample material that would otherwise bind to the vial (e.g. proteins in blood samples). This compound can be a protein (such as albumin) which will take up the surface binding sites in the vial without occupying a significant proportion of the volume.

In one preferred embodiment, the vial contains a substance (either pre-coated on the inner walls of the vial, or as a known volume of solid, liquid, or gas, or impregnated into an absorbent mass affixed to a place inside the vial) that will cause whole blood that comes into contact with the substance to gel in place, thus preventing the separation of plasma from red cells that ordinarily occurs when whole blood is allowed to settle. This allows samples to be counted for an extended period of time with a stable geometry. This is an important consideration when a tracer is present in one component of the blood (e.g. radio-labelled albumin in the plasma, or a fluorescent dye in the plasma, or radio-labelled red cells) and a counting chamber does not have uniform counting efficiency from every part of the vial (for example, because of the opening drilled into a crystal to create a standard counting well, a sample concentrated at the bottom of the well will register slightly more counts than one concentrated near the top of the well).

In another preferred embodiment, dimensionally identical “standard” and “injectate” vials are included as components of a kit to enable an indicator dilution measurement to be performed. A standard vial is prepared by diluting injectate solution (from the same lot used to fill the injectate vials) into a known volume (e.g. 1000 ml). Each injectate vial can be used to perform a separate indicator dilution measurement. Identically dimensioned empty collection vials can be included with such a kit or provided separately. Standards are useful when a detector (such a radiation scintillation detector) does not possess sufficient linearity or range to enable it to count the “hot” injectate directly.

### BRIEF DESCRIPTION OF THE SEVERAL VIEWS OF THE DRAWINGS

FIG. 1A-1D shows one embodiment of the invention, where a precise volume is accessed by an ingress and egress, where the entire assembly is sized and shaped to fit into a counting aperture, for example the well of a gamma scintillation counter.

FIG. 2A-2D shows one embodiment of the invention, where the fluid path has been optimized to place the majority of fluid in the bottom of the vial, where it will be optimally visible to the detector. The cross section of the fluid pathway starts out as a circle (at the ingress, to mate with standard connectors), then transforms continuously to an ellipse, wider across the transverse axis of the tube, to take advantage of the width of the container, then transforms continuously back to a circle (at the egress).

FIG. 3A-3C shows one embodiment of the invention, where the fluid path is defined by extruded tubing within the vial. The tubing forms a coil, concentrating the fluid at the bottom of the vial. Valves are depicted attached to the ingress and egress, allowing convenient connection to other equipment. For example, the ingress could be attached to an IV line in a patient, while the egress could be connected to a syringe. Pulling out the syringe plunger would cause the vial to be filled with blood from the IV line in a flow-through manner.

FIG. 4A-4D shows one embodiment of the invention, where a reservoir of fluid is concentrated at the bottom of the vial. This vial is constructed from two components, an outer sleeve, and an inner dome insert (depicted in FIG. 5A-5D) containing an ingress and egress. The dome insert is fused to the sleeve, resulting in narrow pathways down the sides of the vials leading to a larger volume at the bottom.

FIG. 5A-5D shows the dome insert of the embodiment of FIG. 4A-4D by itself.

FIG. 6 shows one embodiment of the invention, where the vial has been sealed securely with one-way valves so as to transport a quantitative dose of a drug or tracer, prior to its being administered to a patient via connection in a flow-through manner. The vial design from FIG. 1 is utilized. One-way valves indicate the direction of flow (with triangles) by which the contents may be delivered in a flow-through manner.

FIG. 7A-7D shows one embodiment of the invention, where the vial takes a flat form factor. The ingress and egress are located on the side of the vial, and the fluid pathway takes the form of a spiral. This embodiment is designed to be placed on top of a solid detector or be inserted into a slot between two solid detectors. The advantage of this arrangement is very good robustness of measurement with respect to fluid inhomogeneity—no matter where fluid is inside the vial it should experience constant counting geometry. This is particularly important with regard to issues of settling of blood; with this vial, any settling will only occur in the very short transverse dimension and will only alter measurements very slightly.

FIG. 8 shows a vial inserted into the counting well of a detector.

FIG. 9A-9B shows an example of the components of a kit for performing an indicator dilution measurement.

### DETAILED DESCRIPTION OF THE INVENTION

A vial is provided for collecting blood in a flow-through manner, where the vial consists of an internal chamber of precise fixed volume, having an ingress and egress equipped with connectors for standard medical tubing, made of a material with low surface-adhesion properties for proteins, shaped to fit into a counting receptacle such as a cylindrical well, such that the vial can be filled with a precise volume of blood by connecting the ingress with medical tubing to the blood supply of a living subject and applying vacuum pressure at the egress.

The process of manufacture the vial can be injection molding, and the internal volume can consists of a pathway of fixed cross section. The pathway can change shape while maintaining constant cross section, such that the cross section is circular at the ingress and the egress; changes to elliptical in continuous fashion; and the elliptical section is coiled in the vertical plane towards the bottom of the vial so as to concentrate the volume towards the bottom of the vial.

The vial can have a flat spiral, designed to be placed on a flat detector (or in between two flat detectors).

The vial can be made of two moldable components fused together, an outer vial in the shape of a well, and an inner component with a fitted cover with ingress and egress, arranged so that there is a narrow fluid pathway down the sides leading to a larger well at the bottom of the assembled vial.

The vial can be made of one extruded and two moldable components that are fused together: an inner coil, an outer container, and a fitted cover with ingress and egress. The coil can be constructed from a material chosen for its desirable flow properties, particularly low surface adhesion of blood proteins.

The vial can be outfitted with one-way valves at the ingress and egress such that the container can be filled with a precise quantity of a desired fluid (such as a drug or tracer) and securely stored or transported for later administration.

The vial can be pre-treated with a compound such as albumin to alter the surface adhesion properties of the material such that the flow-through performance of the vial is enhanced when exposed to sample material that would otherwise bind to the vial.

The vial can be treated at the time of manufacture with a compound to cause blood in the vial to gel in place rather than settle under the action of gravity.

A lysing membrane can be incorporated across the ingress of the vial, such that cells in the sample do not remain intact.

A kit is provided for performance of an indicator dilution measurement, consisting of a plurality of labelled dimensionally identical vials as disclosed herein provided together in a suitable package. One or more injectate vials can be filled with tracer from a single manufactured lot of tracer. The tracer can be, for example, a radioactive compound or a fluorescent compound. One or more standard vials are filled with a solution created by diluting the same quantity of tracer present in each injectate vial into a known volume. One or more empty sample collection vials can also be provided.

A method is provided for performing an indicator dilution method to determine an unknown volume using any of the kits disclosed herein, whereby

the injectate vial is injected into an unknown volume via flow-through means,

a sample is taken after a short interval from the subject into an empty collection vial, and

the counts of the standard vial and patient vial are used to determine the subject volume.

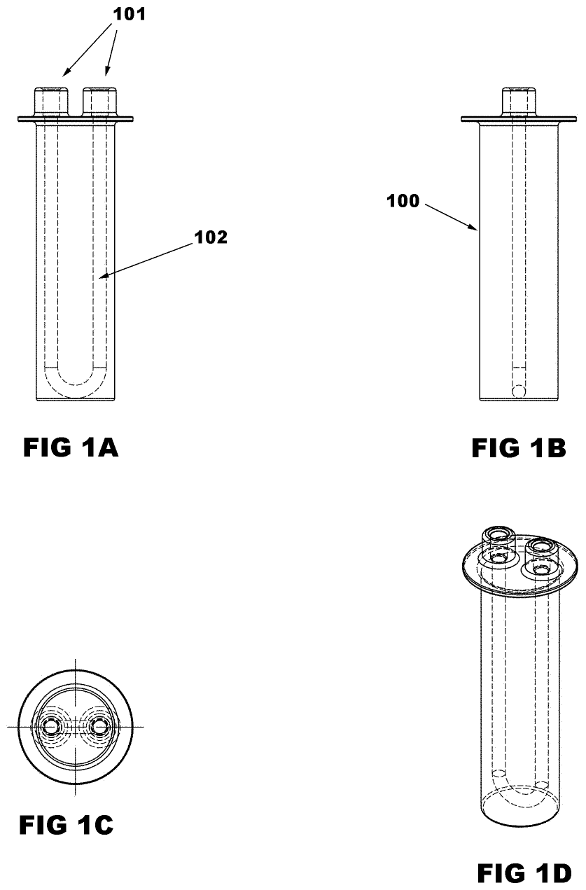

The essential requirements for a quantitative blood collection vial are that it enable collection of a precise amount of blood directly from a subject, that the amount of blood is not limited to the quantity that can be collected by capillary action, that the collection process not require specialized skills beyond those possessed by a phlebotomist, and that the collected sample can immediately be counted in a quantitative detector in the collection vial. FIGS. 1A-1D depict an embodiment of such a vial (100). A simple pathway (102) through the vial is realized through a process such as injection molding. The ingress and egress (101) are fitted with connectors allowing access to standard medical tubing. An example of the use of such an embodiment is to measure the concentration of some tracer (such a radioactive tagged blood product) continuously as it moves through the circulation of a subject. The ingress is connected to an IV line from the patient, and the egress is connected to a source of vacuum (such as a pump, vacutainer tube, or syringe). The vacuum at the egress causes blood to be drawn into the vial, where it can be measured inside a detector at the patient's bedside.

Selection of materials is important for ensuring that blood can be collected reliably. Various materials were tested for their flow-through properties. Materials were tested using whole blood, as this is a very important use case, and because proteins in blood often adhere to plastics, particularly in spaces with small inner diameter. Four different tube materials (as listed in table below) were evaluated by passing a fixed know volume of human whole blood tagged with a radioactive tracer through the tubes. The tubing sections were subsequently flushed with normal saline. Each of the flushed tubes were then measured to determine how much of the blood protein remained bound to the inner surfaces of the tubes. The following table lists the tubing material and the percent of radioactive protein retained.

TABLE 1
Percent retention after flushing of various plastics

| | |
|---|---|
| Flexible Polyethylene (Flexelene) | 1.11% |
| Polyvinyl chloride (PVC) | 1.23% |
| Polypropylene (PET) | 3.52% |
| Fluorinated ethylene propylene (FEP) | 0.49% |

The very low retention value of 0.49% for FEP is most likely due to the ‘non-stick’ qualities imparted by the addition of fluorine to polypropylene. These measurements highlight the significance of fluid and material compatibility when designing a flow through vial.

Gamma scintillation detectors have a geometry effect. When a sample is placed into a counting well, the sample is surrounded on nearly all sides by the detector crystal, with the unavoidable exception of the solid angle subtended by the well opening. The father down the well, the more efficient the counting will be; in the extreme case, a sample placed at the very opening of the well will have approximately half the counting efficiency, ignoring effects of absorbance by the well liner. Therefore, it is desirable to concentrate as much of the sample at the bottom of the well (i.e. at the bottom of the vial). Several embodiments are presented that achieve this objective. Note that the embodiment depicted in FIGS. 1A-1D (which does not concentrate sample toward the bottom of the well) can still achieve precise and accurate counting performance of homogeneous samples so long as dimensionally identical vials are used for counting.

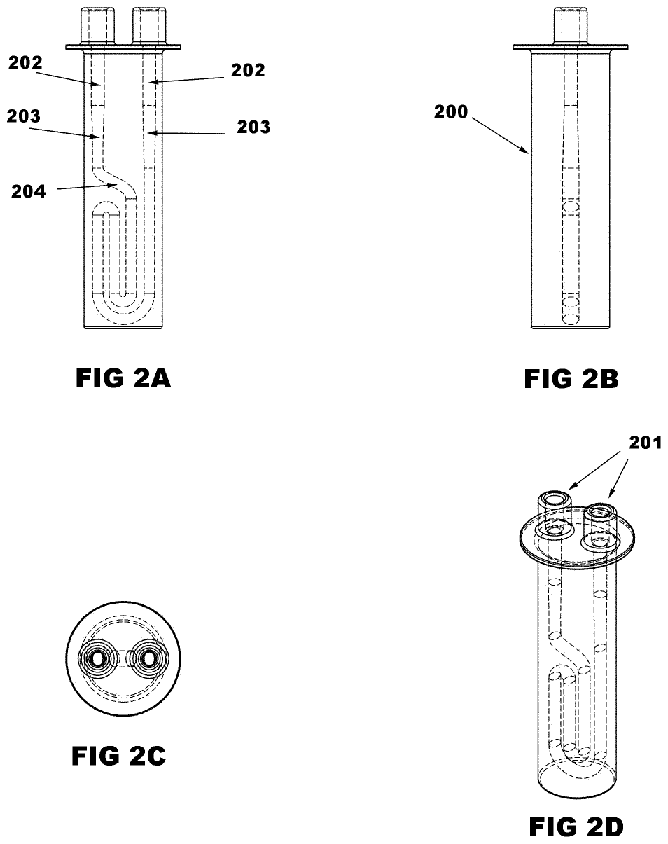

FIGS. 2A-2D show an embodiment where approximately two thirds of the sample to be measured is concentrated in the lower half of the vial. This is achieved by providing a path of constant cross section, but which changes shape. The vial (200) is fitted with an ingress and egress (201). The cross section of the fluid pathway starts out as a circle at the ingress/egress, to mate with standard connectors (202), then transforms continuously (203) to an ellipse (204), wider across the transverse axis of the tube, to take advantage of the width of the container, then transforms continuously (203) back to a circle (202). The constant cross section is an important consideration for flow-through performance, as ideal laminar flow is disturbed by changes in cross section. Since this design has a vertical axis of symmetry, it is manufacturable through a simple process such as injection molding.

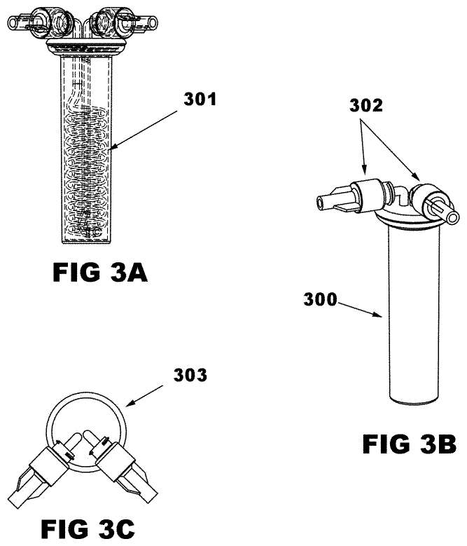

FIGS. 3A-3C show several views of another embodiment of a vial that achieves the aim of having a concentration of volume in the bottom of the well. The vial is made of one extruded and two moldable components that are fused together: an inner coil (301), an outer container (300), and a fitted cover (303) with ingress and egress (302). A coil of extruded tubing (301), made out of a suitable material such as FEP, forms the pathway. The ingress and egress in this figure are shown with one-way valves oriented at right angles for convenience of attachment.

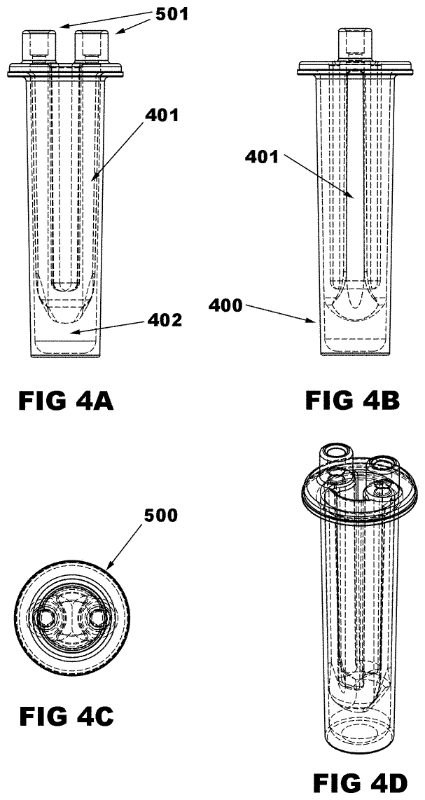

FIGS. 4A-4D show various views of another embodiment that achieves the aim of concentrating sample at the bottom of the well. This embodiment comes quite close to the geometry of a sample sitting in a test tube, in that the ingress and egress tubes hold a small percentage of the total volume of the sample. This vial is constructed from two components, an outer sleeve (400), and an inner dome insert (500) containing an ingress and egress (501). The dome insert is fused to the sleeve, resulting in narrow pathways down the sides of the vials (401) leading to a larger volume at the bottom (402).

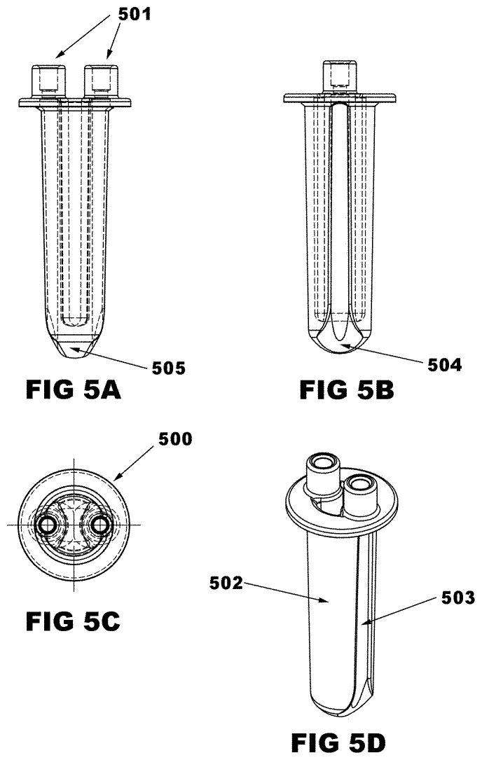

The dome insert (500) with ingress and egress (501) is depicted for clarity on its own in FIGS. 5A-5D. The insert features curved side walls (502) that fuse with the inner wall of the outer sleeve (400), interrupted by channels (503) that connect to the ingress and egress (501). The channels are terminated in a smooth concave form at the bottom (504) to improve flow; similarly, the side walls (502) are terminated at the bottom with a smooth convex form (505).

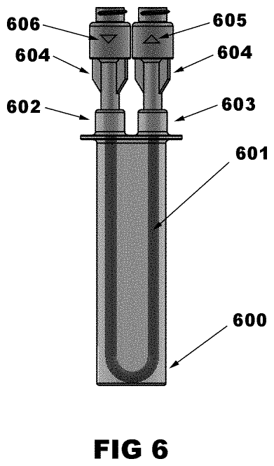

FIG. 6 shows an embodiment where a vial (600) has been prefilled with a precise amount of a fluid (601), e.g. a drug to be administered, or a tracer. The ingress (602) and egress (603) of the vial are sealed with one-way pressure valves (604). The direction of flow is indicated by arrows on the valves, the up-arrow (605) on the egress, and the down-arrow (606) on the ingress. The contents can be administered in a flow-through manner by, for example, connecting the egress of the vial to a patient's IV line via tubing, and connecting a syringe with a suitable volume of saline (i.e. some multiple of the volume of the vial, perhaps 5 to 30 times the volume) to the ingress. The contents are then flushed into the patient with a high degree of accuracy as to the volume delivered, along with saline.

Such a vial could be employed as part of a method for collecting, storing, transporting, and/or measuring a precise volume of blood, comprising the steps of:
a. connecting the ingress of the vial to a source of blood such an IV line,
b. connecting the egress of the vial to a source of vacuum pressure such as a syringe, vacutainer tube, or pump, and
c. applying vacuum pressure to draw blood completely through the vial.

If the vial has sealing mechanisms (such as one-way check valves, pressure valves, stopcocks, etc.) at the ingress and egress as is shown in FIG. 6, the vial can disconnected from the source of blood and source of vacuum after the completion of steps (a) through (c) for more convenient storage, transportation, or measurement at another location.

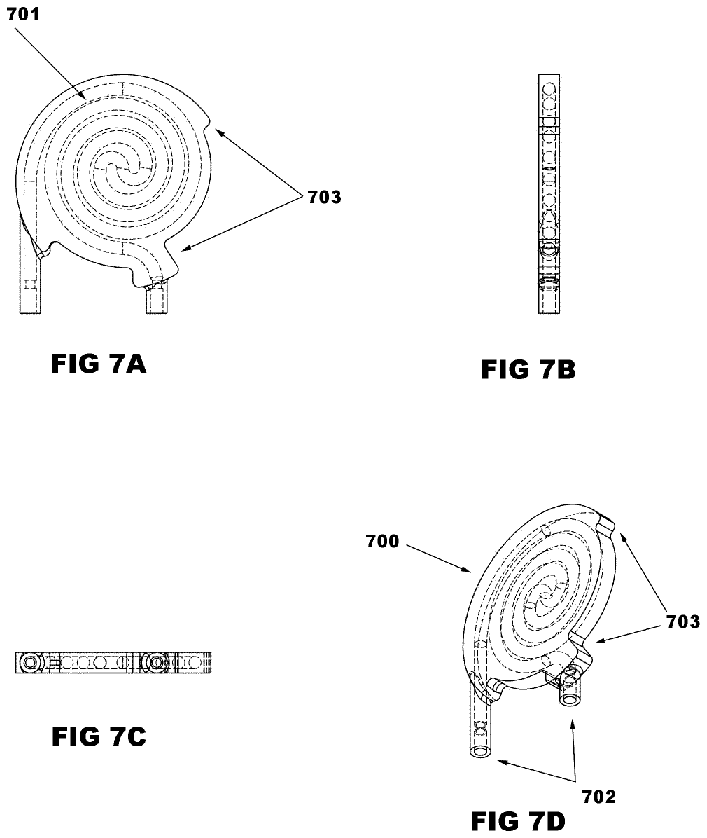

FIGS. 7A-7D show another embodiment, designed to be measured on a flat rather than well detector. This vial (700) design is flat, with a spiral pathway (701) of constant cross section with excellent flow-through properties, which connects the ingress and egress (702). Because the height of the vial is minimal, settling effects on counting will be minimal, especially if a dual detector design is used, i.e. with a flat detector both above and below, with the vial inserted from the side. To ensure consistent counting geometry, the vial is equipped with shaped flanges (703) which can mate with a fitting inside a detector well such that the cartridge seats in the same position every time it is inserted (e.g. by snapping into place). The flat design of this vial is ideal for situations where settling of blood may be an issue (e.g. when there is a long time interval between collection of samples and measurement in an analyzer).

There are also non-geometric methods to deal with issue of settling of blood. In one embodiment, a compound is present in the vial that causes the blood to gel in place rather than settle over time under the influence of gravity. Chitosan and sodium polyacrylate are suitable substances. This compound can be present as a powder or liquid in the vial, or spray-applied during the process of manufacture.

In another embodiment, physical means are used to ensure that the counting sample is homogeneous. A lysing membrane incorporated into the ingress will ensure that red blood cells do not remain intact, which can cut down on settling effects in counting.

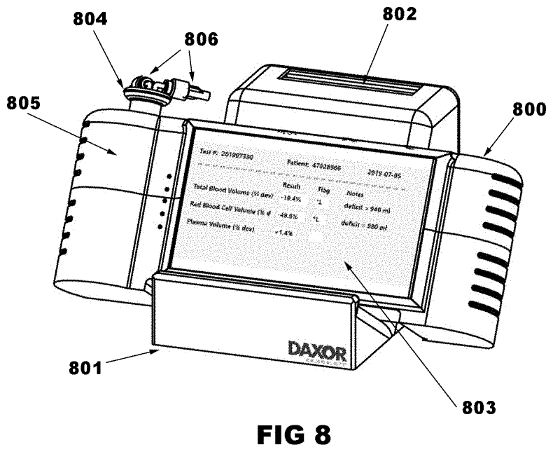

FIG. 8 shows a vial (such as the one depicted in FIGS. 3A-3C) in use, being measured in the counting well of a detector. A handheld analyzer (800) is shown connected to a charging base (801) which includes a printer (802) for printing reports. A touchscreen (803) displays the results of an analysis. The vial (804) is shown inserted into the counting well of a detector (805) integrated into the analyzer. The vial is shown with valves attached to the ingress and egress (806).

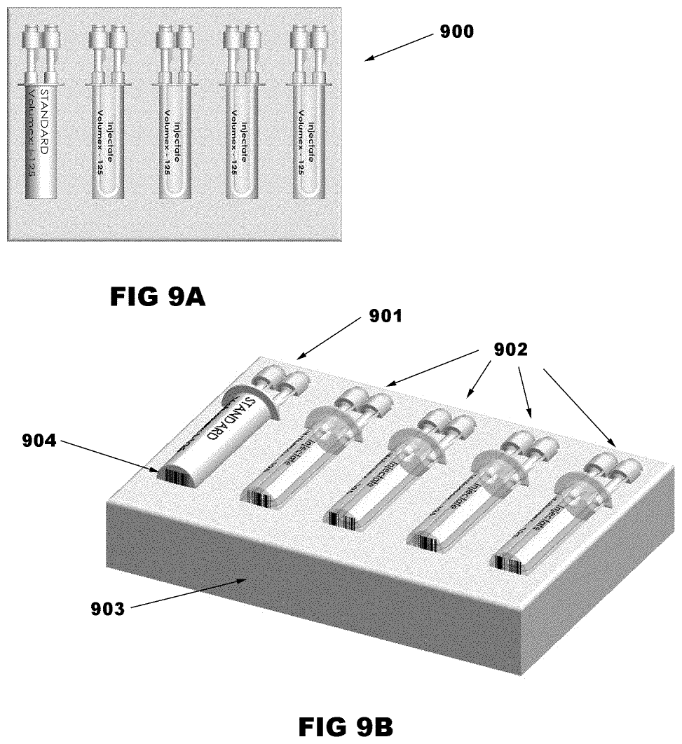

FIGS. 9A-9B shows a preferred embodiment, in which vials are included as components of a kit (900) to enable an indicator dilution measurement to be performed. A set of five filled vials (each of the form depicted in FIG. 6) are provided in a set, packaged in a tray (903) formed of suitable protective material such as cardboard, foam, plastic etc. One vial (901) is marked as a “standard”, the others (902) are injectate vials. The standard vial has been prepared by diluting injectate solution (from the same lot used to fill the injectate vials) into a known volume (e.g. 1000 ml). Each injectate vial (902) can be used to perform a single indicator dilution measurement, with the standard vial (901) being measured but not consumed and hence reusable for multiple measurements. Barcodes (904) on each vial include a unique identifier that ensures that standards and injectate match. Identically dimensioned empty collection vials can be included with such a kit or provided separately. To perform a measurement, an injectate vial is flushed into the unknown volume (e.g. the bloodstream of a living subject). After a short interval (to ensure mixing has occurred), a sample is collected from the subject into an empty collection volume of identical dimensions. By counting the standard vial and the patient vial, the patient volume can be computed using the simple ratio:

$$patient . volume = ( \frac{standard . counts}{patient . counts} ) standard . volume . \qquad ( 1 )$$

One skilled in the art will recognize how background measurements (from patient and room) can be accounted for by subtracting the relevant counts.

What is claimed is:

1. A vial for collecting blood in a flow-through manner, consisting of an internal chamber of precise fixed volume, having an ingress and egress equipped with connectors for standard medical tubing, made of a material with low surface-adhesion properties for proteins, shaped to fit into a counting receptacle such as a cylindrical well, such that the vial can be filled with a precise volume of blood by connecting the ingress with medical tubing to the blood supply of a living subject and applying vacuum pressure at the egress.

2. The vial of claim 1, where the process of manufacture is injection molding, and the internal volume consists of a pathway of fixed cross section.

3. The vial of claim 2, where the pathway changes shape while maintaining constant cross section, such that the cross section is circular at the ingress and the egress; changes to elliptical in continuous fashion; and the elliptical section is coiled in the vertical plane towards the bottom of the vial so as to concentrate the volume towards the bottom of the vial.

4. The vial of claim 2, where the vial is a flat spiral, designed to be placed on a flat detector (or in between two flat detectors).

5. The vial of claim 1, where the vial is made of two moldable components fused together, an outer vial in the shape of a well, and an inner component with a fitted cover with ingress and egress, arranged so that there is a narrow fluid pathway down the sides leading to a larger well at the bottom of the assembled vial.

6. The vial of claim 1, where the vial is made of one extruded and two moldable components that are fused together: an inner coil, an outer container, and a fitted cover with ingress and egress.

7. The vial of claim 6, where the coil is constructed from a material chosen for its desirable flow properties, particularly low surface adhesion of blood proteins.

8. The vial of claim 1, outfitted with one-way valves at the ingress and egress such that the container can be filled with a precise quantity of a desired fluid (such as a drug or tracer) and securely stored or transported for later administration.

9. The vial of claim 1, where the vial is pre-treated with a compound such as albumin to alter the surface adhesion properties of the material such that the flow-through performance of the vial is enhanced when exposed to sample material that would otherwise bind to the vial.

10. The vial of claim 1, where the vial is treated at the time of manufacture with a compound to cause blood in the vial to gel in place rather than settle under the action of gravity.

11. The vial of claim 1, where a lysing membrane is incorporated across the ingress, such that cells in the sample do not remain intact.

12. A kit for performance of an indicator dilution measurement, consisting of a plurality of labelled dimensionally identical vials of claim 1 provided together in a suitable package.

13. The kit of claim 12, where one or more injectate vials are filled with tracer from a single manufactured lot of tracer.

14. The kit of claim 12, where one or more standard vials are filled with a solution created by diluting the same quantity of tracer present in each injectate vial into a known volume.

15. The kit of claim 12, where zero or more empty sample collection vials are also provided.

16. The kit of claim 13, where the tracer is a radioactive compound.

17. The kit of claim 13, where the tracer is a fluorescent compound.

18. A method for collecting, storing, transporting, and/or measuring a precise volume of blood using the vial of claim 1, comprising the steps of:
a. connecting the ingress of the vial to a source of blood such an IV line,
b. connecting the egress of the vial to a source of vacuum pressure such as a syringe, vacutainer tube, or pump, and
c. applying vacuum pressure to draw blood completely through the vial.

19. The method of claim 18, where the vial includes sealing mechanisms (such as one-way check valves, pressure valves, stopcocks, etc.) at the ingress and egress, and the vial is disconnected from the source of blood and source of vacuum after the completion of steps (a) through (c).

20. A method for performing the indicator dilution method to determine an unknown volume using the kit of claim 12, whereby
a. the injectate vial is injected into an unknown volume via flow-through means,
b. a sample is taken after a short interval from the subject into an empty collection vial, and
c. the counts of the standard vial and patient vial are used to determine the subject volume.
